(19) United States
(12) Patent Application Publication
(10) Pub. No.: US 2007/0009463 A1
(43) Pub. Date: Jan. 11, 2007

(54) Rheology profile for a personal care composition

(76) Inventors: Michael Frederick Niebauer, Cincinnati, OH (US); Douglas Allan Royce, Sunman, IN (US); Thomas A. Hutchins, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/480,151

(22) Filed: Jun. 30, 2006

Related U.S. Application Data
(60) Provisional application No. 60/696,926, filed on Jul. 6, 2005.

(51) Int. Cl. A61K 8/02

(52) U.S. Cl. 424/70.7; 424/401

(57) ABSTRACT

Personal care compositions comprising a desirable rheology profile. A method of delivering benefits to hair or skin is provided.

### CROSS REFERENCE TO RELATED APPLICATION

This application claims the benefit of U.S. Provisional Application No. 60/696,926 filed Jul. 6, 2005.

### FIELD

The present invention relates to personal care compositions having a rheology profile that has a high viscosity at low shear rates and a lower viscosity at higher shear rates. The present invention further relates to a multi-phase personal care composition comprising at least two phases with at least one of the phases wherein the phases are visually distinct phases that are packaged in physical contact and maintain stability.

### BACKGROUND

Personal care compositions are well known and widely used. These compositions have long been employed to cleanse and moisturize skin, condition the hair, deliver actives, hide imperfections, and to reduce the oiliness/shine associated with sebum. Personal care compositions have also been used to alter the color and appearance of skin.

While the compositions and disclosures of the prior art provide useful advances in the art of personal care compositions, additionally, there remains the need for improved personal care compositions that provide multiple benefits. One attempt at providing multiple benefits from a personal care product has been the dual-chamber packaging. These packages comprise separate compositions and allow for co-dispensing of the two in a single or dual stream. The separate compositions remain physically separate and stable during prolonged storage and just prior to application, then mix during or after dispensing to provide multiple benefits from a physically stable system. Although such dual-chamber delivery systems provide improved consumer benefits over the use of conventional systems, it is often difficult to achieve consistent and uniform performance because of the uneven dispensing ratio between the cleansing phase and the benefit phase from these dual chamber packages. Additionally, these packaging systems add considerable cost to the finished product.

Another approach to provide multiple benefits from a single product has been to use multi-phase compositions. However, in order to maintain stability of the multiple phases in these compositions it has typically been necessary to formulate these products to have a gel-like consistency. It would be desirable to provide a personal care composition providing multiple benefits wherein the composition behaves more like typical shampoos or other single phase personal care compositions. Accordingly, the need still remains for personal care compositions that provide multiple benefits delivered from products having the stability, look, and feel of single phase compositions.

### SUMMARY

One embodiment of the present invention relates to a personal care composition having a rheology profile substantially corresponding to the following:

a) a viscosity in the range of from about 25 Pas to about 2000 Pas at a low-shear rate of from about 0.0005 s⁻¹ to about 0.005⁻¹ at about 25° C.;

b) a shear rate index of less than about 1 at a shear rate of from about 0.01 s⁻¹ to about 1.0 s⁻¹ at about 25° C.; and

c) a viscosity in the range of from about 0 Pas to about 50 Pas at a high-shear rate of from about 10 s⁻¹ to about 1000 s⁻¹ at about 25° C.

Another embodiment of the present invention relates to a multi-phase personal care composition wherein at least one phase exhibits a rheology profile substantially corresponding to the following:

a) a viscosity in the range of from about 25 Pas to about 2000 Pas at a low-shear rate of from about 0.0005 s⁻¹ to about 0.005 s⁻¹ at about 25° C.;

b) a shear rate index of less than about 1 at a shear rate of from about 0.01 s⁻¹ to about 1.0 s⁻¹ at about 25° C.; and

c) a viscosity in the range of from about 0 Pas to about 50 Pas at a high-shear rate of from about 10 s⁻¹ to about 1000 s⁻¹ at about 25° C.; and wherein said at least two phases are visually distinct phases that are packaged in physical contact and maintain stability.

In accordance with yet another aspect of the invention, a method of delivering benefits to hair or skin is provided. The method includes topically applying an effective amount of a single-phase or multi-phase composition having the improved rheology profile described herein onto said hair or skin and removing the composition from said hair or skin by rinsing with water.

### DETAILED DESCRIPTION

The personal care compositions of the present invention exhibit a Non-Newtonian, shear thinning rheology profile characterized by relatively high viscosity at low shear rates and relatively low viscosity at high shear rates. These and other limitations of the compositions and methods of the present invention, as well as many of the optional ingredients suitable for use herein, are described in detail hereinafter.

The essential components of the personal care composition are described below. Also included is a nonexclusive description of various optional and preferred components useful in embodiments of the present invention. While the specification concludes with claims that particularly point out and distinctly claim the invention, it is believed the present invention will be better understood from the following description.

All percentages, parts, and ratios are based upon the total weight of the compositions of the present invention, unless otherwise specified. All such weights as they pertain to listed ingredients are based on the active level and, therefore do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

The term “ambient conditions,” as used herein, unless otherwise specified, refers to surrounding conditions at one (1) atmosphere of pressure, 50% relative humidity, and 25° C.

The term “anhydrous,” as used herein, unless otherwise specified, refers to those compositions or materials containing less than about 10%, more preferably less than about 5%, even more preferably less than about 3%, even more preferably zero percent, by weight of water.

The term “charge density,” as used herein, refers to the ratio of the number of positive charges on a monomeric unit of which a polymer is comprised to the molecular weight of said monomeric unit. The charge density multiplied by the polymer molecular weight determines the number of positively charged sites on a given polymer chain.

The term “Consistency value,” or “k,” as used herein, is a measure of viscosity and is used in combination with Shear Index, to define viscosity for materials whose viscosity is a function of shear. The measurements are made at 25° C. and the units are poise (equal to 100 cps).

By the term “multi-phased,” or “multi-phase,” as used herein, is meant that at least two phases occupy separate and distinct physical spaces inside the package in which they are stored, but are in direct contact with one another (i.e., they are not separated by a barrier and they are not emulsified). In one preferred embodiment of the present invention, the “multi-phased” personal care compositions comprising at least two phases are present within the container as a visually distinct pattern. The pattern results from the mixing or homogenization of the “multi-phased” composition. The patterns include but are not limited to the following examples: striped, marbled, rectilinear, interrupted striped, checked, mottled, veined, clustered, speckled, geometric, spotted, ribbons, helical, swirled, arrayed, variegated, textured, grooved, ridged, waved, sinusoidal, spiraled, twisted, curved, cycle, streaked, striated, contoured, anisotropic, laced, weaved or woven, basket weave, spotted, and tessellated. Preferably the pattern is selected from the group consisting of striped, geometric, marbled, and combinations thereof. In a preferred embodiment the striped pattern may be relatively uniform and even across the dimension of the package. Alternatively, the striped pattern may be uneven, i.e., wavy, or may be non-uniform in dimension. The striped pattern does not need to necessarily extend across the entire dimension of the package. The phases may be various different colors, or include particles, glitter or pearlescence.

The term “personal care composition,” as used herein, unless otherwise specified, refers to the compositions of the present invention, wherein the compositions are intended to include only those compositions for topical application to the hair or skin, and specifically excludes those compositions that are directed primarily to other applications such as hard surface cleansing, fabric or laundry cleansing, and similar other applications not intended primarily for topical application to the hair or skin.

The term “Shear Index,” or “n,” as used herein, is a measure of viscosity and is used to define viscosity for materials whose viscosity is a function of shear. The Power law model is used to determine a rate index (shear stress=viscosity*shear rate shear index). If the index is equal to 1, it is Newtonian; if the index is greater than 1, it is shear thickening; if the index is less than 1, it is shear thinning. The measurements are made at 25° C. and the units are dimensionless.

The term “stable,” as used herein, unless otherwise specified, refers to compositions in which the visible pattern or arrangement of the phases in different locations in the package is not significantly changing overtime when sitting in physical contact at ambient conditions for a period of at least about 180 days. In addition, it is meant that no separation, creaming, or sedimentation occurs. By “separation” is meant that the well-distributed nature of the visually distinct phases is compromised, such that larger regions of at least one phase collect until the balanced dispensed ratio of the two or more compositions relative to each other is compromised.

By the term “visibly clear,” as used herein, is meant that the transmission of the composition is greater than 60%, preferably greater than 80%. The transparency of the composition is measured using Ultra-Violet/Visible (UV/VIS) Spectrophotometry, which determines the absorption or transmission of UV/VIS light by a sample. A light wavelength of 600 nm has been shown to be adequate for characterizing the degree of clarity of cosmetic compositions. Typically, it is best to follow the specific instructions relating the specific spectrophotometer being used. In general, the procedure for measuring percent transmittance starts by setting the spectrophotometer to the 600 nm. Then a calibration “blank” is run to calibrate the readout to 100 percent transmittance. The test sample is then placed in a cuvette designed to fit the specific spectrophotometer and the percent transmittance is measured by the spectrophotometer at 600 nm.

By the term “visually distinct,” as used herein, is meant that the regions occupied by each phase can be separately seen by the human eye as distinctly separate regions in contact with one another (i.e., they are not emulsions or dispersions of particles of less than about 100 microns). The distinction can be as a result of, for example, color or texture or transmittance variations.

In a particular embodiment, the visually distinct phases are of a different color, or one phase may be visibly clear. For instance, one or more phases can comprise a dye, pigment, pearlescent agent, lake, coloring, or mixtures thereof. Colorants useful in the present invention can be, for example, selected from the group consisting of Red 30 Low Iron, FD&C Red 40 AL Lake, D&C Red Lake Blend of Lake 27 & Lake 30, FD&C Yellow 5 Al Lake, FD&C Yellow 6 Al Lake, FD&C Yellow 5 Lake, FD&C Blue #1 AL Lake, Kowet Titanium Dioxide, D&C Red 30 Talc Lake, D&C Red 6 Barium Lake, D&C Red 7 Calcium Lake, D&C Red 34 Calcium Lake, D&C Red 30! AL lake, D&C Red 27 AL lake, D&!; C Yellow 10 AL lake, D&C Red 21 AL Lake, Yellow Iron Oxide, D&C Red 30 Lake, Octocir Yellow 6 AL Lake, Octocir Yellow 5 AL Lake, D&C Red 28 Lake, D&C Orange 5 Zirc Al Lake, Cos Red Oxide BC, Cos Iron Oxide Red BC, Cos Iron oxide Black BC, Cos Iron Oxide Yellow, Cos Iron Oxide Brown, Cos Iron Oxide Yellow BC, Euroxide Red Unsteril, Euroxide Black Unsteril, Euroxide Yellow Steril, Euroxide Black Steril, Euroxide Red, Euroxide Black, Hydrophobic Euroxide Black, Hydrophobic Euroxide Yellow, Hydrophobic Euroxide Red, D&C Yellow 6 Lake, D&C Yellow 5 Zr Lake, and mixtures thereof.

The term “water soluble,” as used herein, means that the component is soluble in water in the present composition. In general, the component should be soluble at about 25° C. at a concentration of about 0.1% by weight of the water solvent, preferably at about 1%, more preferably at about 5%, even more preferably at about 15%.

The personal care compositions and methods of the present invention can comprise, consist of, or consist essentially of, the essential elements and limitations of the invention described herein, as well as any additional or optional ingredients, components, or limitations described herein or otherwise useful in personal care compositions intended for topical application to the hair or skin.

The present invention relates to personal care compositions that exhibit a Non-Newtonian, shear thinning rheology profile characterized by relatively high viscosity at low shear rates and relatively low viscosity at high shear rates. The compositions of the present invention preferably have a pH of from about 2 to about 8.5, more preferably from about 3 to about 7.5, even preferably from about 3.5 to about 6.5.

The personal care compositions of the present invention may include, but are not limited to, shampoo, conditioner, antiperspirant, deodorant, hair styling products, cleansers, soaps, bar soap, bodywash, cosmetics, foundations, lotions, creams, ointments, and hydro-alcoholic solutions.

The personal care composition may comprise multiple phases. These products are intended to provide a multi-phase personal care composition that is packaged in physical contact while remaining stable and providing improved in-use and after use hair and skin benefits. The phases may be one or a combination of a cleansing phase, a conditioning phase, a benefit phase, or the like. Further, one or more of the phases may also comprise additional components. In the present invention, one or more of the phases may be visibly clear.

The multi-phase personal care compositions of the present invention comprise at least two phases, wherein the composition can have a first phase, a second phase and so on. The ratio of a first phase to a second phase may be from about 99:1 to about 1:99, from about 95:5 to about 5:95, from about 90:10 to about 10:90, preferably from about 80:20 to about 20:80, more preferably from about 70:30 to about 30:70, even more preferably from about 60:40 to about 40:60, still more preferably about 50:50.

A. Rheology Profile

The composition, or at least one phase of the composition, has a preferred rheology profile as defined by viscosity (η) and Shear Index (η). The phase can be characterized by viscosity (η) and Shear Index (η) values as defined below, wherein these defined ranges are selected to provide enhanced deposition and reduced stickiness during and after application of the personal cleaning composition on hair or skin.

The Shear Index (η) and viscosity (η) values are well known and accepted industry standards for reporting the viscosity profile of materials having a viscosity that is a function of an applied shear rate.

The viscosity (η) for any material can be characterized by the relationship or equation
[η=σ/γ′]
wherein σ is shear stress and γ′ is shear rate, so that the viscosity for any material can be measured by either applying a shear rate and measuring the resultant shear stress or vice versa.

An AR2000 Rheometer, available from TA Instruments of New Castle, Del., USA, is used to determine Shear Index, n, and Consistency value, k, for the phases herein. The determination is performed at 25° C. with the 4 cm 2° cone (truncated to 51μ) measuring system set with a 51 micron gap and is performed via the programmed application of a shear stress (typically from about 0.06 dynes/sq. cm to about 5,000 dynes/sq. cm) over time. These data are used to create a viscosity (η) versus shear rate (γ′) flow curve for the phases. This flow curve can then be modeled in order to provide a mathematical expression that describes the material's behavior within specific limits of shear stress and shear rate. These results are fitted with the following well-accepted power law model (see for instance: Chemical Engineering, by Coulson and Richardson, Pergamon, 1982 or Transport Phenomena by Bird, Stewart and Lightfoot, Wiley, 1960):
[η=k(γ′)ⁿ⁻¹]

where “k” is the “Consistency value” which is used in combination with Shear Index, to define viscosity for materials whose viscosity is a function of shear.

The composition, or at least one phase of the composition exhibits a relatively high viscosity at low shear, a relatively low viscosity at high shear and is highly shear thinning. More particularly the composition or at least one phase of the composition exhibits a rheology profile substantially corresponding to the following:

a viscosity in the range of from about 25 Pas to about 2000 Pas, preferably from about 50 Pas to about 1500 Pas, at a low-shear rate of from about 0.0005 s⁻¹ to about 0.005 s⁻¹;

a shear rate index of less than about 1, preferably in the range of from about 0.20 to about 0.75, more preferably in the range of from about 0.30 to about 0.50, at a shear rate of from about 0.01 s⁻¹ to about 1.0 s⁻¹; and

a viscosity in the range of from about 0 to about 50 Pas, preferably from about 0 Pas to about 20 Pas, more preferably from about 1 Pas to about 15 Pas, still more preferably from about 1 Pas to about 10 Pas, at a high-shear rate of from about 10 s⁻¹ to about 1000 s⁻¹.

B. Components

The personal care composition, or at least one phase of the personal care composition, of the present invention may comprise various components, including, but not limited to, surfactants, thickener, cationic surfactants, fatty compound.

1. Surfactants

The personal care compositions of the present invention may comprise at least one surfactant. Suitable surfactants for use herein include any known or otherwise effective cleansing surfactant which is suitable for application to the hair or skin, and which are otherwise compatible with the other essential ingredients of the compositions. The surfactants useful herein may be selected from the group consisting of anionic surfactants, nonionic surfactants, zwitterionic surfactants, amphoteric surfactants, cationic surfactants, soap, and mixtures thereof. Suitable surfactants are described in McCutcheon's, Emulsifiers and Detergents, 1989 Annual, published by M. C. Publishing Co., and in U.S. Pat. No. 3,929,678.

The surfactant may be present at concentrations ranging from about 4% to about 50%, more preferably from about 9% to about 30%. The surfactant may be present in an amount of at least 4% by weight of the composition, preferably at least about 9%.

Preferred anionic surfactants include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium tridecyl benzene sulfonate, sodium dodecyl benzene sulfonate, and combinations thereof.

Anionic surfactants with branched alkyl chains such as sodium trideceth sulfate, for example, are preferred in some embodiments. Mixtures of anionic surfactants can be used in some embodiments.

Zwitterionic surfactants suitable for use as cleansing surfactant include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate.

Nonionic surfactants for use in the compositions of the present invention are disclosed in McCutcheon's, Detergents and Emulsifiers, North American edition (1986), published by allured Publishing Corporation; and McCutcheon's, Functional Materials, North American Edition (1992).

Nonionic surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Nonionic lathering surfactants also useful herein include, lauramine oxide, cocoamine oxide.

Other suitable non-ionic surfactants are Surfadone LP-100 and Surfadone LP-300 from International Specialty Products.

2. Thickener

The present invention may comprise at least one thickener. Preferred thickeners are selected from the group consisting of inorganic water thickeners, polymeric thickeners, additives that promote thickening via lamellar structuring of surfactants, organic crystalline thickeners, and mixtures thereof.

The thickener in the present invention can be hydrophilic. The amount of thickener present may be less than about 20%, preferably less than about 10%, and even more preferably less than about 5%.

Non-limiting examples of inorganic water thickeners for use in the personal care composition include silicas, clays such as a synthetic silicates (Laponite XLG and Laponite XLS from Southern Clay), or mixtures thereof.

Non-limiting examples of polymeric thickeners for use in the personal care composition include Acrylates/Vinyl Isodecanoate Crosspolymer (Stabylen 30 from 3V), Acrylates/C10-30 Alkyl Acrylate Crosspolymer (Pemulen TR1 and TR2), Carbomers (Aqua SF-1), Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant), Acrylates/Ceteth-20 Itaconate Copolymer (Structure 3001 from National Starch), Polyacrylamide (Sepigel 305 from SEPPIC), Non-ionic thickener, (Aculyn 46 from Rohm and Haas), or mixtures thereof.

Additional non-limiting examples of polymeric thickeners for use in the personal care composition include cellulosic gel, hydroxypropyl starch phosphate (Structure XL from National Starch), polyvinyl alcohol, hydroxyethyl cellulose, or mixtures thereof.

Further, non-limiting examples of polymeric thickeners for use in the personal care composition include synthetic and natural gums and thickeners such as xanthan gum (Ketrol CG-T from CP Kelco), succinoglycan (Rheozan from Rhodia), gellum gum, pectin, alginates, starches including pregelatinized starches, modified starches, or mixtures thereof, acrylates/aminoacrylates/CD-30 alkyl PEG-20 itaconate copolymer (Structure Plus from National Starch).

3. Cationic Surfactant

The personal care compositions of the present invention may comprise a cationic surfactant. The cationic surfactant is included in the composition at a level by weight of preferably from about 0.1% to about 10%, more preferably from about 1% to about 8%, still more preferably from about 2% to about 5%.

The cationic surfactant, together with below high melting fatty compound, and an aqueous carrier, may provide a gel matrix which is suitable for providing various conditioning benefits such as slippery feel on wet hair and softness and moisturized feel on dry hair. In view of providing the above gel matrix, the cationic surfactant and the high melting point fatty compound are contained at a level such that the mole ratio of the cationic surfactant to the high melting point fatty compound is in the range of, preferably from about 1:1 to 1:10, more preferably from about 1:2 to 1:6.

Preferred cationic surfactants are those having a longer alkyl group, i.e., C18-22 alkyl group. Such cationic surfactants include, for example, behenyl trimethyl ammonium chloride and stearyl trimethyl ammonium chloride, and still more preferred is behenyl trimethyl ammonium chloride. It is believed that cationic surfactants having a longer alkyl group provide improved deposition on the hair, thus can provide improved conditioning benefits such as improved softness on dry hair, compared to cationic surfactant having a shorter alkyl group. It is also believed that such cationic surfactants can provide reduced irritation, compared to cationic surfactants having a shorter alkyl group.

Nonlimiting examples of cationic surfactants useful in the present invention include the materials having the following CTFA designations: quaternium-8, quaternium-14, quaternium-18, quaternium-18 methosulfate, quaternium-24, and mixtures thereof.

4. Fatty Compound

The composition may comprise a fatty compound, which may consist of from about 0.01% to about 20%, preferably from about 0.1% to about 15%, more preferably from about 0.2% to about 10%, by weight of the composition. A gel matrix may be formed by the fatty compound, and/or the cationic surfactant compound may be first mixed with, suspended in, and/or dissolved in water when forming a gel matrix.

The fatty compound useful herein has a melting point of 25° C. or higher and is selected from the group consisting of fatty alcohols, fatty acids, and mixtures thereof. It is understood that the compounds disclosed in this section of the specification can in some instances fall into more than one classification, e.g., some fatty alcohol derivatives may also be classified as fatty acid derivatives. However, a given classification is not intended to be a limitation on that particular compound, but is done so for the convenience of classification and nomenclature. Further, it is understood that depending on the number and position of double bonds and length and position of branches, certain compounds having certain required carbon atoms may have a melting point of less than 25° C. Such compounds of low melting point are not intended to be included in this section. Nonlimiting examples of high melting compounds are found in International Cosmetic Ingredient Dictionary, Fifth Edition, 1993, and CTFA Cosmetic Ingredient handbook, Second Edition, 1992.

The fatty alcohols useful herein are those having from about 14 to about 30 carbon atoms, preferably from about 16 to about 22 carbon atoms. These fatty alcohols are saturated and can be straight or branched chain alcohols. Nonlimiting examples of fatty alcohols include cetyl alcohol, stearyl alcohol, behenyl alcohol, and mixtures thereof.

The fatty acids useful herein are those having from about 10 to about 30 carbon atoms, preferably from about 12 to about 25 carbon atoms, and more preferably from about 16 to about 22 carbon atoms. These fatty acids are saturated and can be straight or branched chain acids. Also included are diacids, triacids, and other multiple acids that meet the requirements herein. Also included herein are the salts of these fatty acids. Nonlimiting examples of fatty acids include lauric acid, palmitic acid, stearic acid, behenic acid, sebacic acid, and mixtures thereof.

Fatty compounds of a single compound of high purity are preferred. Single compounds of pure fatty alcohols selected from the group of pure cetyl alcohol, stearyl alcohol, and behenyl alcohol are preferred. By “pure” herein, what is meant is that the compound has a purity of at least about 90%, preferably at least about 95%. These single compounds of high purity may provide good rinseability from the hair when the consumer rinses off the composition.

5. Benefit Agents

The composition may comprise a benefit agent selected from the group consisting of styling polymers, silicones, crosslinked silicone elastomers, peralkylene hydrocarbons, and hair coloring agents/dyes, anti-dandruff actives, humectants, water soluble nonionic polymers, cationic polymers, conditioning agents, and particles.

a. Styling Polymers

The compositions of the present invention may comprise a styling polymer. The compositions hereof will generally comprise from about 0.1% to about 15%, preferably from 0.5% to about 8%, more preferably from about 1% to about 8%, by weight of the composition, of the styling polymer. It is not intended to exclude the use of higher or lower levels of the polymers, as long as an effective amount is used to provide adhesive or film-forming properties to the composition and the composition can be formulated and effectively applied for its intended purpose.

These styling polymers provide the composition of the present invention with hair styling performance by providing polymeric deposits on the hair after application. The polymer deposited on the hair has adhesive and cohesive strength and delivers styling primarily by forming welds between hair fibers upon drying, as is understood by those skilled in the art.

Many such polymers are known in the art, including water-soluble and water-insoluble organic polymers and water-insoluble silicone-grafted polymers, all of which are suitable for use in the composition herein, provided that they also have the requisite features or characteristics described hereinafter. Such polymers can be made by conventional or otherwise known polymerization techniques well known in the art, an example of which includes free radical polymerization.

A wide variety of natural, semi-natural, and synthetic styling polymers are useful herein, see suitable styling polymers in encyclopedia of polymers and thickeners, Cosmetic & Toiletries, Volume 117, No. 12, December 2002, pages 67-120.

b. Silicones

The compositions of the present invention may comprise a silicone. The silicone is preferably an insoluble silicone conditioning agent. The silicone conditioning agent particles may comprise volatile silicone, non-volatile silicone, or combinations thereof. Preferred are non-volatile silicone conditioning agents. If volatile silicones are present, it will typically be incidental to their use as a solvent or carrier for commercially available forms of non-volatile silicone materials ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair.

The concentration of the silicone conditioning agent typically ranges from about 0.01% to about 10%, preferably from about 0.1% to about 8%, more preferably from about 0.1% to about 5%, more preferably from about 0.2% to about 3%. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609. The silicone conditioning agents for use in the compositions of the present invention preferably have a viscosity, as measured at 25° C., from about 20 to about 2,000,000 centistokes (“csk”), more preferably from about 1,000 to about 1,800,000 csk, even more preferably from about 50,000 to about 1,500,000 csk, more preferably from about 100,000 to about 1,500,000 csk.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989).

Silicone resins may be included in the silicone conditioning agent of the compositions of the present invention. These resins are highly cross-linked polymeric siloxane systems. The cross-linking is introduced through the incorporation of trifunctional and tetrafunctional silanes with monofunctional or difunctional, or both, silanes during manufacture of the silicone resin.

c. Crosslinked Silicone Elastomers

The personal care compositions of the present invention may comprise crosslinked silicone elastomers. Crosslinked silicone elastomers are present in an amount of from about 0.01% to about 15%, preferably from about 0.1% to about 10%, even more preferably from about 1% to about 5% by weight of the composition. These benefit agents provide hair alignment and softness (emollient) benefits to hair. Preferred compositions are dimethicone/vinyl dimethicone crosspolymers. Such dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer]), Grant Industries (Gransil™ line of materials), and lauryl dimethicone/vinyl dimethicone crosspolymers supplied by Shin Etsu (e.g., KSG-31, KSG-32, KSG-41, KSG-42, KSG-43, and KSG-44). Cross-linked organopolysiloxane elastomers useful in the present invention and processes for making them are further described in U.S. Pat. No. 4,970,252; U.S. Pat. No. 5,760,116; U.S. Pat. No. 5,654,362; and Japanese Patent Application JP 61-18708, assigned to Pola Kasei Kogyo KK. Silicone elastomers of the type described in U.S. Pat. Nos. 5,412,004; 5,837,793; and 5,811,487, are also useful herein. Preferably the elastomers of the present invention are cured under anhydrous conditions or in an anhydrous environment.

d. Peralkylene Hydrocarbons

The present invention may include peraklylene hydrocarbon materials. These materials are a branched alk(en)yl material, of which the side-groups are —H, C₁₋₄ alk(en)yl groups or (—H or C₁₋₄ alk(en)yl) substituted saturated or unsaturated cyclic hydrocarbons, and wherein at least 10% by number of the side-groups are other than —H, more preferably from 25% to 75%, most preferably from 40% to 60%. Preferred alkyl side-groups are methyl groups.

Preferably the weight average molecular weight of the per-alk(en)yl hydrocarbon material is less than about 4200, preferably from about 180 to about 2500. Such low molecular weight per-alk(en)yl hydrocarbon materials are available for example from BP under the trade name Indopol, from Soltex under the tradename Solanes and from Chevron under the tradename Oronite OLOA.

It is also advantageous to control the particle size of the per-alk(en)yl hydrocarbon materials in order to maintain suitable conditioning characteristic of the composition. The combination of per-alk(en)yl hydrocarbon materials having a particle size from about 0.01μ to about 40μ and cationic deposition polymers, especially celluloses, allow for the conditioning aspects of the formula to be controlled and targeted towards a given consumer group. Through the use of low molecular weight per-alk(en)yl hydrocarbon materials, the need for large levels of expensive conditioning oils to mitigate the trade-offs traditionally associated with styling shampoos is significantly reduced.

Preferred per-alk(en)yl hydrocarbon materials are polymers of butene, isoprene, terpene and styrene, and copolymers of any combination of these monomers, such as butyl rubber (poly isobutylene-co-isoprene), natural rubber (cis-1,4-polyisoprene) and hydrocarbon resins such as mentioned in the Encyclopedia of Chemical Technology by Kirk & Ohmer (3rd edition vol 8, pp 852-869), for example aliphatic and aromatic petroleum resins, terpene resins etc. Especially preferred is the use of polymers which are soluble in the low molecular weight per-alk(en)yl hydrocarbon material or other solvent or carrier, if used.

Examples of particularly useful materials are available from Presperse Inc. under the Permethyl trade name. The total level of per-alk(en)yl hydrocarbon material in the hair styling composition is preferably from about 0.01% to about 10%, more preferably from about 0.2% to about 5% even more preferably from about 0.2% to about 2% by weight of the composition.

e. Hair Coloring Agents/Dyes

The compositions of the present invention may also include hair coloring agents/dyes. Hair coloring agents/dyes useful herein include anthroquinone, azo, nitro, basic, triarylmethane, or disperse dyes, or any combinations thereof. A range of direct dyes, including basic dyes and neutral dyes are useful herein. Dyes suitable for use are described in U.S. Pat. No. 5,281,240 and U.S. Pat. No. 4,964,874.

f. Anti-Dandruff Actives

The compositions of the present invention may also contain an anti-dandruff agent. Suitable, non-limiting examples of anti-dandruff particulates include: pyridinethione salts, azoles, selenium sulfide, climbazole, particulate sulfur, and mixtures thereof. Preferred are pyridinethione salts. Such anti-dandruff particulate should be physically and chemically compatible with the essential components of the composition, and should not otherwise unduly impair product stability, aesthetics or performance.

Pyridinethione anti-dandruff particulates, especially 1-hydroxy-2-pyridinethione salts, are highly preferred particulate anti-dandruff agents for use in compositions of the present invention. The concentration of pyridinethione anti-dandruff particulate typically ranges from about 0.1% to about 4%, by weight of the composition, preferably from about 0.1% to about 3%, more preferably from about 0.3% to about 2%. Preferred pyridinethione salts include those formed from heavy metals such as zinc, tin, cadmium, magnesium, aluminum and zirconium, preferably zinc, more preferably the zinc salt of 1-hydroxy-2-pyridinethione (known as “zinc pyridinethione” or “ZPT”), more preferably 1-hydroxy-2-pyridinethione salts in platelet particle form, wherein the particles have an average size of up to about 20μ, preferably up to about 5μ, more preferably up to about 2.5μ. Salts formed from other cations, such as sodium, may also be suitable. Pyridinethione anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982. It is contemplated that when ZPT is used as the anti-dandruff particulate in the compositions herein, that the growth or re-growth of hair may be stimulated or regulated, or both, or that hair loss may be reduced or inhibited, or that hair may appear thicker or fuller.

In addition to the anti-dandruff active selected from polyvalent metal salts of pyrithione, the present invention may further comprise one or more anti-fugal or anti-microbial actives in addition to the metal pyrithione salt actives. Suitable anti-microbial actives include coal tar, sulfinur, whitfield's ointment, castellani's paint, aluminum chloride, gentian violet, octopirox (piroctone olamine), ciclopirox olamine, undecylenic acid and it's metal salts, potassium permanganate, selenium sulphide, sodium thiosulfate, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Preferred anti-microbials include itraconazole, ketoconazole, selenium sulphide and coal tar.

Azole anti-microbials include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. When present in the composition, the azole anti-microbial active is included in an amount from about 0.01% to about 5%, preferably from about 0.1% to about 3%, and more preferably from about 0.3% to about 2%, by weight of the composition. Especially preferred herein is ketoconazole.

Selenium sulfide is a particulate anti-dandruff agent suitable for use in the compositions of the present invention, effective concentrations of which range from about 0.1% to about 4%, by weight of the composition, preferably from about 0.3% to about 2.5%, more preferably from about 0.5% to about 1.5%. Selenium sulfide is generally regarded as a compound having one mole of selenium and two moles of sulfur, although it may also be a cyclic structure that conforms to the general formula SeₓS_{y}, wherein x+y=8. Average particle diameters for the selenium sulfide are typically less than 15 μm, as measured by forward laser light scattering device (e.g., Malvern 3600 instrument), preferably less than 10 μm. Selenium sulfide compounds are described, for example, in U.S. Pat. No. 2,694,668; U.S. Pat. No. 3,152,046; U.S. Pat. No. 4,089,945; and U.S. Pat. No. 4,885,107.

Sulfur may also be used as a particulate anti-microbial/anti-dandruff agent in the anti-microbial compositions of the present invention. Effective concentrations of the particulate sulfur are typically from about 1% to about 4%, by weight of the composition, preferably from about 2% to about 4%.

The present invention may further comprise one or more keratolytic agents such as Salicylic Acid.

Additional anti-microbial actives of the present invention may include extracts of melaleuca (tea tree) and charcoal. The present invention may also comprise combinations of anti-microbial actives. Such combinations may include octopirox and zinc pyrithione combinations, pine tar and sulfur combinations, salicylic acid and zinc pyrithione combinations, octopirox and climbasole combinations, and salicylic acid and octopirox combinations, and mixtures thereof.

g. Humectants

The compositions of the present invention may contain a humectant. The humectants herein are selected from the group consisting of polyhydric alcohols, water soluble alkoxylated nonionic polymers, and mixtures thereof. The humectants are preferably used at levels of from about 0.1% to about 20%, more preferably from about 0.5% to about 5%.

Polyhydric alcohols useful herein include glycerin, sorbitol, propylene glycol, butylene glycol, hexylene glycol, ethoxylated glucose, 1,2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof.

Water soluble alkoxylated nonionic polymers useful herein include polyethylene glycols and polypropylene glycols having a molecular weight of up to about 1000 such as those with CTFA names PEG-200, PEG-400, PEG-600, PEG-1000, and mixtures thereof.

h. Water Soluble Nonionic Polymers

The compositions of the present invention may comprise from about 0.1% to about 10%, more preferably from about 0.2% to about 5%, and even more preferably from about 0.5% to about 3% by weight of a water soluble nonionic polymer.

The polymers of the present invention are characterized by the general formula: 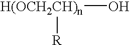
wherein R is selected from the group consisting of H, methyl, and mixtures thereof. When R is H, these materials are polymers of ethylene oxide, which are also known as polyethylene oxides, polyoxyethylenes, and polyethylene glycols. When R is methyl, these materials are polymers of propylene oxide, which are also known as polypropylene oxides, polyoxypropylenes, and polypropylene glycols. When R is methyl, it is also understood that various positional isomers of the resulting polymers can exist. In the above structure, n has an average value of from about 2,000 to about 14,000, preferably from about 5,000 to about 9,000, more preferably from about 6,000 to about 8,000.

Polyethylene glycol polymers useful herein that are especially preferred are PEG-2M wherein R equals H and n has an average value of about 2,000 (PEG 2-M is also known as Polyox WSR® N-10 from Union Carbide and as PEG-2,000); PEG-5M wherein R equals H and n has an average value of about 5,000 (PEG 5-M is also known as Polyox WSR® N-35 and Polyox WSR® N-80, both from Union Carbide and as PEG-5,000 and Polyethylene Glycol 300,000); PEG-7M wherein R equals H and n has an average value of about 7,000 (PEG 7-M is also known as Polyox WSR® N-750 from Union Carbide); PEG-9M wherein R equals H and n has an average value of about 9,000 (PEG 9-M is also known as Polyox WSR® N-3333 from Union Carbide); and PEG-14 M wherein R equals H and n has an average value of about 14,000. (PEG 14-M is also known as Polyox WSR® N-3000 from Union Carbide.) Other useful polymers include the polypropylene glycols and mixed polyethylene/polypropylene glycols.

i. Cationic Polymers

The compositions of the present invention may contain a cationic polymer. Concentrations of the cationic polymer in the composition typically are less than about 3%, preferably less than about 2.0%, more preferably less than about 0.1%. Preferred cationic polymers will have cationic charge densities of at least about 0.7 meq/gm, preferably at least about 1.2 meq/gm, more preferably at least about 1.5 meq/gm, but also preferably less than about 7 meq/gm, more preferably less than about 5 meq/gm, at the pH of intended use of the composition, which pH will generally range from about pH 3 to about pH 9, preferably between about pH 4 and about pH 8. Herein, “cationic charge density” of a polymer refers to the ratio of the number of positive charges on the polymer to the molecular weight of the polymer. The average molecular weight of such suitable cationic polymers will generally be between about 10,000 and 10 million, preferably between about 50,000 and about 5 million, more preferably between about 100,000 and about 3 million.

Suitable cationic polymers for use in the compositions of the present invention contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the composition. Any anionic counterions can be used in association with the cationic polymers so long as the polymers remain soluble in water, in the composition, or in a coacervate phase of the composition, and so long as the counterions are physically and chemically compatible with the essential components of the composition or do not otherwise unduly impair product performance, stability or aesthetics. Non limiting examples of such counterions include halides (e.g., chloride, fluoride, bromide, iodide), sulfate and methylsulfate.

Non limiting examples of such polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)).

Non limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone.

Suitable cationic protonated amino and quaternary ammonium monomers, for inclusion in the cationic polymers of the composition herein, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Other suitable cationic polymers for use in the compositions include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16); copolymers of 1-vinyl-2-pyrrolidone and dimethylaminoethyl methacrylate (referred to in the industry by CTFA as Polyquaternium-1); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquaternium 6 and Polyquaternium 7, respectively); amphoteric copolymers of acrylic acid including copolymers of acrylic acid and dimethyldiallylammonium chloride (referred to in the industry by CTFA as Polyquaternium 22), terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride and methylacrylate (referred to in the industry by CTFA as Polyquaternium 47). Preferred cationic substituted monomers are the cationic substituted dialkylaminoalkyl acrylamides, dialkylaminoalkyl methacrylamides, and combinations thereof.

Other suitable cationic polymers for use in the composition include polysaccharide polymers, such as cationic cellulose derivatives and cationic starch derivatives. Preferred cationic cellulose polymers are salts of hydroxyethyl cellulose reacted with trimethyl ammonium substituted epoxide, referred to in the industry (CTFA) as Polyquaternium 10 and available from Amerchol Corp. (Edison, N.J., USA) in their Polymer LR, JR, and KG series of polymers. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp. under the tradename Polymer LM-200.

Other suitable cationic polymers include cationic guar gum derivatives, such as guar hydroxypropyltrimonium chloride, specific examples of which include the Jaguar series commercially avaialable from Rhone-Poulenc Incorporated and the N-Hance series commercially available from Aqualon Division of Hercules, Inc. Other suitable cationic polymers include quaternary nitrogen-containing cellulose ethers, some examples of which are described in U.S. Pat. No. 3,962,418. Other suitable cationic polymers include copolymers of etherified cellulose, guar and starch, some examples of which are described in U.S. Pat. No. 3,958,581. When used, the cationic polymers herein are either soluble in the composition or are soluble in a complex coacervate phase in the composition formed by the cationic polymer and the anionic, amphoteric and/or zwitterionic detersive surfactant component described hereinbefore. Complex coacervates of the cationic polymer can also be formed with other charged materials in the composition.

Techniques for analysis of formation of complex coacervates are known in the art. For example, microscopic analyses of the compositions, at any chosen stage of dilution, can be utilized to identify whether a coacervate phase has formed. Such coacervate phase will be identifiable as an additional emulsified phase in the composition. The use of dyes can aid in distinguishing the coacervate phase from other insoluble phases dispersed in the composition.

j. Particles

The personal care composition of the present invention may comprise particles. Water insoluble solid particle of various shapes and densities is useful. The particle of the present invention has a particle size (volume average based on the particle size measurement described hereafter) of less than about 100 μm, preferably less than about 60 μm, and more preferably the particle size of less than about 30 μm.

The particles that can be present in the present invention can be natural, synthetic, or semi-synthetic. In addition, hybrid particles can also be present. Synthetic particles can made of either cross-linked or non cross-linked polymers. The particles of the present invention can have surface charges or their surface can be modified with organic or inorganic materials such as surfactants, polymers, and inorganic materials. Particle complexes can be present.

Nonlimiting examples of synthetic particles include nylon, silicone resins, poly(meth)acrylates, polyethylene, polyester, polypropylene, polystyrene, polyurethane, polyamide, epoxy resins, urea resins, and acrylic powders. Non limiting examples of useful particles are Microease 110S, 114S, 116 (micronized synthetic waxes), Micropoly 210, 250S (micronized polyethylene), Microslip (micronized polytetrafluoroethylene), and Microsilk (combination of polyethylene and polytetrafluoroethylene), all of which are available from Micro Powder, Inc. Additional examples include Luna (smooth silica particles) particles available from Phenomenex, MP-2200 (polymethylmethacrylate), EA-209 (ethylene/acrylate copolymer), SP-501(nylon-12), ES-830 (polymethly methacrylate), BPD-800, BPD-500 (polyurethane) particles available from Kobo Products, Inc. and silicone resins sold under the name Tospearl particles by GE Silicones. Ganzpearl GS-0605 crosslinked polystyrene (available from Presperse) is also useful.

Non limiting examples of hybrid particles include Ganzpearl GSC-30SR (Sericite & crosslinked polystyrene hybrid powder), and SM-1000, SM-200 (mica and silica hybrid powder available from Presperse).

The interference pigments of the present invention are platelet particulates. The platelet particulates of the multi-phased personal care compositions preferably have a thickness of no more than about 5 μm, more preferably no more than about 2 μm, still more preferably no more than about 1 μm. The platelet particulates of the multi-phased personal care composition preferably have a thickness of at least about 0.02 μm, more preferably at least about 0.05 μm, even more preferably at least about 0.1 μm, and still more preferably at least about 0.2 μm.

The interference pigment of the multi-phased personal care compositions comprise a multilayer structure. The centre of the particulates is a flat substrate with a refractive index (RI) normally below 1.8. A wide variety of particle substrates are useful herein. Nonlimiting examples are natural mica, synthetic mica, graphite, talc, kaolin, alumina flake, bismuth oxychloride, silica flake, glass flake, ceramics, titanium dioxide, CaSO₄, CaCO₃, BaSO₄, borosilicate and mixtures thereof, preferably mica, silica and alumina flakes.

A layer of thin film or a multiple layer of thin films are coated on the surface of a substrate described above. The thin films are made of highly refractive materials. The refractive index of these materials is normally above 1.8.

A wide variety of thin films are useful herein. Nonlimiting examples are TiO₂, Fe₂O₃, SnO₂, Cr₂O₃, ZnO, ZnS, ZnO, SnO, ZrO₂, CaF₂, Al₂O₃, BiOCl, and mixtures thereof or in the form of separate layers, preferably TiO₂, Fe₂O₃, Cr₂O₃SnO₂. For the multiple layer structures, the thin films can be consisted of all high refractive index materials or alternation of thin films with high and low RI materials with the high RI film as the top layer.

Nonlimiting examples of the interference pigments useful herein include those supplied by Persperse, Inc. under the trade name PRESTIGE®, FLONAC®; supplied by EMD Chemicals, Inc. under the trade name TIMIRON®, COLORONA®, DICHRONA® and XIRONA®; and supplied by Engelhard Co. under the trade name FLAMENCO®, TIMICA®, DUOCHROME®.

In an embodiment of the present invention the interference pigment surface is either hydrophobic or has been hydrophobically modified. The Particle Contact Angle Test as described in copending application Ser. No. 60/469,075 filed on May 8, 2003 is used to determine contact angle of interference pigments. The greater the contact angle, the greater the hydrophobicity of the interference pigment. The interference pigment of the present invention possess a contact angle of at least 60 degrees, more preferably greater than 80 degrees, even more preferably greater than 100 degrees, still more preferably greater than 100 degrees.

Nonlimiting examples of the hydrophobic surface treatment useful herein include silicones, acrylate silicone copolymers, acrylate polymers, alkyl silane, isopropyl titanium triisostearate, sodium stearate, magnesium myristate, perfluoroalcohol phosphate, perfluoropolymethyl isopropyl ether, lecithin, carnauba wax, polyethylene, chitosan, lauroyl lysine, plant lipid extracts and mixtures thereof, preferably, silicones, silanes and stearates. Surface treatment houses include US Cosmetics, KOBO Products Inc., and Cardre Inc.

6. Additional Phases

The present invention may comprise a high viscosity aqueous phase that may comprise a water thickener and water. The high viscosity aqueous phase can be hydrophilic. In a preferred embodiment the high viscosity aqueous phase is a hydrophilic gelled water phase. In addition, the high viscosity aqueous phase of the present invention may comprise less than about 5%, preferably less than about 3%, and more preferably less than about 1%, by weight of the high viscosity aqueous phase, of a surfactant. In one embodiment of the present invention, the high viscosity aqueous phase is free of surfactant.

The present invention may also comprise a high viscosity oil-in-water high internal phase emulsion (HIPE) comprising an oil and an aqueous carrier. The phase may also comprise a stabilizer. The high internal phase emulsion is an emulsion containing about 50% or more of a discontinuous or “internal” phase and about 50% or less of a continuous phase. The oil phase is the discontinuous phase and the aqueous phase is the continuous phase.

C. Aqueous Carrier

The compositions of the present invention may comprise an aqueous carrier. The aqueous carrier may be found in one or both phases of the composition. Preferably, they comprise from about 50% to about 99.8%, by weight of water. The aqueous carrier can optionally include other liquid, water-miscible or water-soluble solvents such as lower alkyl alcohols, e.g., C₁-C₅ alkyl monohydric alcohols, preferably C₂-C₃ alkyl alcohols.

D. Optional Ingredients

The compositions herein can contain a variety of other optional components suitable for rendering such compositions more cosmetically or aesthetically acceptable or to provide them with additional usage benefits. Optional ingredients may be found in either phase. Such conventional optional ingredients are well-known to those skilled in the art.

A wide variety of additional ingredients can be formulated into the present composition. These include: other conditioning agents; viscosity modifiers such as alkanolamides and methanolamides of long chain fatty acids such as cocomonoethanol amide; crystalline suspending agents; pearlescent aids such as ethylene glycol distearate; preservatives such as benzyl alcohol, methyl paraben, propyl paraben and imidazolidinyl urea; polyvinyl alcohol; ethyl alcohol; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents, such as the thioglycolates; perfumes; sequestering agents, such as disodium ethylenediamine tetra-acetate; and polymer plasticizing agents, such as glycerin, disobutyl adipate, butyl stearate, and propylene glycol. Other non limiting examples of these optional ingredients include vitamins and derivatives thereof (e.g., ascorbic acid, vitamin E, tocopheryl acetate, and the like); sunscreens; thickening agents (e.g., polyol alkoxy ester, available as Crothix from Croda); preservatives for maintaining the anti microbial integrity of the cleansing compositions; anti-acne medicaments (resorcinol, salicylic acid, and the like); antioxidants; skin soothing and healing agents such as aloe vera extract, allantoin and the like; chelators and sequestrants; and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol).

Other optional hair and skin benefit ingredients include carboxylic acid which is hydroxylated in the a position (which compound is also referred to as an α—(alpha) hydroxyl acid) or a derivative thereof. Acid derivatives, as defined herein, are associated salts (salts with organic bases or alkali metal, for example) or lactides (obtained, for example, by autiesterification of a-hydroxy acid molecules). Examples of such compounds are, citric acid, lactic acid, methallactic acid, phenyllactic acid, malic acid, mandelic acid, glycolic acid, benzylic acid, and 2-hydroxycaprylic acid.

Additional hair and skin benefit agents include ceramides or glycoceramides. Ceramides are described in Arch. Dermatol, Vol 123, 1381-1384, 1987, or those described in French Patent No. FR-2,673,179; fatty acid polyesters such as, sucrose pentalaurate, sucrose tetraoleate, sucrose pentaerucate, sucrose tetraerucate, sucrose pentatallowate, sucrise triapeate, sucrose tetrapeate, sucrose pentarapeate, sucrose tristearate, and sucrose pentastearate, and mixtures thereof; polypeptides and amino acids consisting of basic amino acids, particularly arginine.

The compositions optionally comprise a colorant or pigment. Preferably, the colorant comprises metal ions. The colorants for use in the compositions are selected from the group consisting of organic pigments, inorganic pigments, interference pigments, lakes, natural colorants, pearlescent agents, dyes, carmines, and mixtures thereof. Non-limiting examples of colorants include: D&C Red 30 Talc Lake, D&C Red 7 Calcium Lake, D&C Red 34 Calcium Lake, Mica/Titanium Dioxide/Carmine Pigments (Clorisonne Red from Engelhard, Duocrome RB from Engelhard, Magenta from Rona, Dichrona RB from Rona), Red 30 Low Iron, D&C Red Lake Blend of Lake 27 & Lake 30, FD&C Yellow 5 Lake, Kowet Titanium Dioxide, Yellow Iron Oxide, D&C Red 30 Lake, D&C Red 28 Lake, Cos Red Oxide BC, Cos Iron Oxide Red BC, Cos Iron oxide Black BC, Cos Iron Oxide Yellow, Cos Iron Oxide Brown, Cos Iron Oxide Yellow BC, Euroxide Red Unsteril, Euroxide Black Unsteril, Euroxide Yellow Steril, Euroxide Black Steril, Euroxide Red, Euroxide Black, Hydrophobic Euroxide Black, Hydrophobic Euroxide Yellow, Hydrophobic Euroxide Red, D&C Yellow 6 Lake, D&C Yellow 5 Zr Lake, and mixtures of these colorants.

### Method of Use

The personal care compositions of the present invention are used in conventional ways to provide cleansing, conditioning and/or other benefits. Such method of use depends upon the type of composition employed but generally involves application of an effective amount of the product to the hair or skin, which may then be rinsed from the hair or skin (as in the case of hair rinses) or allowed to remain on the hair or skin (as in the case of gels, lotions, and creams). “Effective amount” means an amount sufficient enough to provide a dry combing benefit. In general, from about 1 g to about 50 g is applied to the hair, skin, or the scalp. The composition is distributed throughout the hair or skin, typically by rubbing or massaging the hair, scalp, or skin. Preferably, the composition is applied to wet or damp hair prior to drying of the hair. The composition may optionally be applied via a substrate. After such compositions are applied to the hair, the hair is dried and styled in accordance with the preference of the user. In the alternative, the composition is applied to dry hair, and the hair is then combed or styled in accordance with the preference of the user. The personal care compositions are useful in delivering conditioning benefits to hair or skin, and/or delivering hair styling benefits to hair or skin, and/or delivering hair coloring benefits to hair or skin by topically applying an effective amount of the composition onto hair or skin and removing said composition from said hair or skin by rinsing with water.

### Method of Making

The personal care compositions of the present invention may be prepared by any known or otherwise effective technique, suitable for making and formulating the desired striped product form. It is especially effective to combine toothpaste-tube filling technology with a spinning stage design. Specific non-limiting examples of such methods as they are applied to specific embodiments of the present invention are described in the following examples.

### Non-Limiting Examples

The compositions illustrated in the following Examples exemplify specific embodiments of the compositions of the present invention, but are not intended to be limiting thereof. Other modifications can be undertaken by the skilled artisan without departing from the spirit and scope of this invention. Phases A and B are prepared separately.

The compositions illustrated in the following Examples are prepared by conventional formulation and mixing methods, an example of which is described above. All exemplified amounts are listed as weight percents and exclude minor materials such as diluents, preservatives, color solutions, imagery or conceptual ingredients, botanicals, and so forth, unless otherwise specified.

Examples 1-10 - Multi-phase composition

| | | | | | | | | | |
|---|---|---|---|---|---|---|---|---|---|
| 1 | 2 | 3 | 4 | 5 | 6 | 7 | 8 | 9 | 10 |

Phase A Composition

| | | | | | | | | | | |
|---|---|---|---|---|---|---|---|---|---|---|
| Ammonium Laureth-3 Sulfate | 10 | | | 10 | | | 6 | 6 | 8 | |
| Ammonium Lauryl Sulfate | 6 | | | 4 | | | 10 | 10 | 10 | |
| Sodium Laureth-3 Sulfate | | 8 | 6 | | 6 | 10 | | | | 10 |
| Sodium Lauryl Sulfate | | 8 | 10 | | 10 | 6 | | | | 7 |
| Cocamidopropyl Betaine FB | | | | 1 | | | | | | |
| Sodium Lauraoamphoacetate | | | | 1 | | | | | | |
| Cocamide MEA | 0.80 | 0.80 | 1.50 | 0.80 | 0.80 | 0.80 | 1.35 | 1.35 | 0.80 | 0.80 |
| Ethylene Glycol Distearate | 1.00 | 0 | 1.50 | 1.50 | 1.50 | 1.00 | 0 | 1.50 | 1.50 | 0 |
| Cetyl Alcohol | 0.6 | | 0.9 | | | | | | | 0.45 |
| Polyquat 10 (1) | 0.1 | | 0.1 | | 0.25 | | | 0.25 | 0.05 | 0.1 |
| Guar Hydroxypropyl trimonium | | 0.25 | | 0.05 | | | 0.2 | | | |
| Chloride (2) | | | | | | | | | | |
| Polymethacrylamidopropyl trimonium | | | | | | 0.4 | | | 0.2 | |
| Chloride (3) | | | | | | | | | | |
| Carbopol Aqua SF-1 (4) | 1.5 | 3 | 2.5 | 2 | 2 | 2.75 | 1.75 | 2.5 | 1.5 | 2 |
| PEG 14M (5) | 0.1 | | 0.05 | 0.15 | | 0.05 | 0.1 | 0.05 | 0.1 | 0.1 |
| Dimethicone (6) | 2.00 | 0 | 1.5 | 1.00 | 2.00 | 2.00 | 0 | 2.00 | 2.00 | 0 |
| Perfume Solution | 0.8 | 0.8 | 0.8 | 0.8 | 0.8 | 0.8 | 0.8 | 0.8 | 0.8 | 0.8 |
| Citric Acid | 0.4 | 0.9 | 0.8 | 0.8 | 0.8 | 0.8 | 0.4 | 0.4 | 0.4 | 0.8 |
| Sodium Hydroxide | 0.35 | 0.5 | 0.4 | 0.35 | 0.35 | 0.5 | 0.35 | 0.5 | 0.35 | 0.35 |
| Ammonium Xylene Sulfonate | | | | | | | | 1 | | |
| Sodium Xylene Sulfonate | | 1.5 | 0.5 | | | 0.75 | | | | |
| Water and Minors (QS to 100%) | | | | | | | | | | |

Phase B Composition

| | | | | | | | | | | |
|---|---|---|---|---|---|---|---|---|---|---|
| Ammonium Laureth-3 Sulfate | 10 | | | 10 | | | 6 | 6 | 8 | |
| Ammonium Lauryl Sulfate | 6 | | | 4 | | | 10 | 10 | 10 | |
| Sodium Laureth-3 Sulfate | | 8 | 6 | | 6 | 10 | | | | 10 |
| Sodium Lauryl Sulfate | | 8 | 10 | | 10 | 6 | | | | 7 |
| Cocamidopropyl Betaine FB | | | | 1 | | | | | | |
| Sodium Lauraoamphoacetate | | | | 1 | | | | | | |
| Cocamide MEA | 0.80 | 0.80 | 1.50 | 0.80 | 0.80 | 0.80 | 1.35 | 1.35 | 0.80 | 0.80 |
| Ethylene Glycol Distearate | 1.00 | 1.50 | 1.50 | 1.50 | 1.50 | 1.00 | 1.50 | 1.50 | 1.50 | 1.25 |
| Cetyl Alcohol | 0.6 | | 0.9 | | | | | | | 0.45 |
| Polyquat 10 (1) | 0.1 | | 0.1 | | 0.25 | | | 0.25 | 0.05 | 0.1 |
| Guar Hydroxypropyl trimonium | | 0.25 | | 0.05 | | | 0.2 | | | |
| Chloride (2) | | | | | | | | | | |
| Polymethacrylamidopropyl trimonium | | | | | | 0.4 | | | 0.2 | |
| Chloride (3) | | | | | | | | | | |
| Carbopol Aqua SF-1 (4) | 1.5 | 3 | 2.5 | 2 | 2 | 2.75 | 1.75 | 2.5 | 1.5 | 2 |
| PEG 14M (5) | 0.1 | | 0.05 | 0.15 | | 0.05 | 0.1 | 0.05 | 0.1 | 0.1 |
| Dimethicone (6) | 2.00 | 3 | 1.5 | 1.00 | 2.00 | 2.00 | 3.75 | 2.00 | 2.00 | 5 |
| Perfume Solution | 0.8 | 0.8 | 0.8 | 0.8 | 0.8 | 0.8 | 0.8 | 0.8 | 0.8 | 0.8 |
| Citric Acid | 0.4 | 0.9 | 0.8 | 0.8 | 0.8 | 0.8 | 0.4 | 0.4 | 0.4 | 0.8 |
| Sodium Hydroxide | 0.35 | 0.5 | 0.4 | 0.35 | 0.35 | 0.5 | 0.35 | 0.5 | 0.35 | 0.35 |
| Ammonium Xylene Sulfonate | | | | | | | | 1 | | |
| Sodium Xylene Sulfonate | | 1.5 | 0.5 | | | 0.75 | | 0 | | |
| Water and Minors (QS to 100%) | | | | | | | | | | |
| Ratio of Phase A to Phase B | 70:30 | 67:33 | 50:50 | 10:90 | 33:67 | 90:10 | 67:33 | 60:40 | 90:10 | 60:40 |

(1) Polymer LR30M available from Amerchol/Dow Chemical
(2) Jaguar C17 available from Rhodia
(3) Polycare 133 available from Rhodia
(4) Carbopol Aqua SF-1 available from Noveon
(5) PEG14M, WSR N-3000 available from Union Carbide/Dow Chemical
(6) Viscasil 330M available from General Electric Silicones

Examples 11-20 - Single Phase Composition

| | 11 | 12 | 13 | 14 | 15 | 16 | 17 | 18 | 19 | 20 |
|---|---|---|---|---|---|---|---|---|---|---|
| Ammonium Laureth-3 Sulfate | 10 | | | 10 | | | 6 | 6 | 8 | |
| Ammonium Lauryl Sulfate | 6 | | | 4 | | | 10 | 10 | 10 | |
| Sodium Laureth-3 Sulfate | | 8 | 6 | | 6 | 10 | | | | 10 |
| Sodium Lauryl Sulfate | | 8 | 10 | | 10 | 6 | | | | 7 |
| Cocamidopropyl Betaine FB | | | | 1 | | | | | | |
| Sodium Lauraoamphoacetate | | | | 1 | | | | | | |
| Cocamide MEA | 0.80 | 0.80 | 1.50 | 0.80 | 0.80 | 0.80 | 1.35 | 1.35 | 0.80 | 0.80 |
| Ethylene Glycol Distearate | 1.00 | 0 | 1.50 | 1.50 | 1.50 | 1.00 | 0 | 1.50 | 1.50 | 0 |
| Cetyl Alcohol | 0.6 | | 0.9 | | | | | | | 0.45 |
| Polyquat 10 (1) | 0.1 | | 0.1 | | 0.25 | | | 0.25 | 0.05 | 0.1 |
| Guar Hydroxypropyl trimonium | | 0.25 | | 0.05 | | | 0.2 | | | |
| Chloride (2) | | | | | | | | | | |
| Polymethacrylamidopropyl trimonium | | | | | | 0.4 | | | 0.2 | |
| Chloride (3) | | | | | | | | | | |
| Carbopol Aqua SF-1 (4) | 1.5 | 3 | 2.5 | 2 | 2 | 2.75 | 1.75 | 2.5 | 1.5 | 2 |
| PEG 14M (5) | 0.1 | | 0.05 | 0.15 | | 0.05 | 0.1 | 0.05 | 0.1 | 0.1 |
| Dimethicone (6) | 2.00 | 0 | 1.5 | 1.00 | 2.00 | 2.00 | 0 | 2.00 | 2.00 | 0 |
| Perfume Solution | 0.8 | 0.8 | 0.8 | 0.8 | 0.8 | 0.8 | 0.8 | 0.8 | 0.8 | 0.8 |
| Citric Acid | 0.4 | 0.9 | 0.8 | 0.8 | 0.8 | 0.8 | 0.4 | 0.4 | 0.4 | 0.8 |
| Sodium Hydroxide | 0.35 | 0.5 | 0.4 | 0.35 | 0.35 | 0.5 | 0.35 | 0.5 | 0.35 | 0.35 |
| Ammonium Xylene Sulfonate | | | | | | | | 1 | | |
| Sodium Xylene Sulfonate | | 1.5 | 0.5 | | | 0.75 | | | | |
| Water and Minors (QS to 100%) | | | | | | | | | | |

(1) Polymer LR30M available from Amerchol/Dow Chemical
(2) Jaguar C17 available from Rhodia
(3) Polycare 133 available from Rhodia
(4) Carbopol Aqua SF-1 available from Noveon
(5) PEG14M, WSR N-3000 available from Union Carbide/Dow Chemical
(6) Viscasil 330M available from General Electric Silicones

For compositions utilizing Carbopol Aqua SF-1 acylate thickeners, in an appropriate vessel, add surfactants and water. Stir at an appropriate speed using an appropriate sized stir blade. Add citric acid to the formula, and then add the anionic polymer and stir to wet and disperse. Next add NaOH to the mixture. While continuing to stir, adjust the pH using citric acid and NaOH to reduce the pH. Add sodium benzoate, EDTA, water, nonionic polymer, and cationic polymer. Adjust temperature to 55-75° C. and add CMEA. Cool to room temperature. While stirring add dimethicone, D&C pigment, and perfume.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Background, Summary of the Invention, and Detailed Description of the Invention are, in relevant part, incorporated herein by reference; the citation of any document is not to be construed as an admission that it is prior art with respect to the present invention.

What is claimed is:

1. A personal care composition having a rheology profile substantially corresponding to the following:
a.) a viscosity in the range of from about 25 Pas to about 2000 Pas at a low-shear rate of from about 0.0005 s⁻¹ to about 0.005 s⁻¹ at about 25° C.;
b.) a shear rate index of less than about 1 at a shear rate of from about 0.01 s⁻¹ to about 1.0 s⁻¹ at about 25° C.; and
c.) a viscosity in the range of from about 0 Pas to about 50 Pas at a high-shear rate of from about 10 s⁻¹ to about 1000 s⁻¹ at about 25° C.

2. A multi-phase personal care composition wherein at least one phase exhibits a rheology profile substantially corresponding to the following:
a.) a viscosity in the range of from about 25 Pas to about 2000 Pas at a low-shear rate of from about 0.0005 s⁻¹ to about 0.005 s⁻¹ at about 25° C.;
b.) a shear rate index of less than about 1 at a shear rate of from about 0.01 s⁻¹ to about 1.0 s⁻¹ at about 25° C.; and
c.) a viscosity in the range of from about 0 Pas to about 50 Pas at a high-shear rate of from about 10 s⁻¹ to about 1000 s⁻¹ at about 25° C.

3. The multi-phase personal care composition of claim 2 wherein said multi-phase personal care composition has at least two visually distinct phases.

4. The multi-phase personal care composition of claim 2, wherein at least one of said phases is visibly clear.

5. The multi-phase personal care composition of claim 2 wherein said multi-phase personal care composition forms a pattern selected from the group consisting of striped, geometric, marbled, and combinations thereof.

6. The multi-phase personal care composition of claim 2, wherein said phases are packaged in physical contact with one another and maintain stability.

7. The multi-phase personal care composition of claim 2, wherein said shear rate index is in the range of from about 0.20 to about 0.75.

8. The multi-phase personal care composition of claim 2, wherein said shear rate index is in the range of from about 0.30 to about 0.50.

9. The multi-phase personal care composition of claim 2, wherein said viscosity at said low shear rate is in the range of from about 50 Pas to about 1500 Pas.

10. The multi-phase personal care composition of claim 2, wherein said viscosity at said high shear rate is in the range of from about 0 Pas to about 20 Pas.

11. The multi-phase personal care composition of claim 2, wherein said viscosity at said high shear rate is in the range of from about 1 Pas to about 15 Pas.

12. The multi-phase personal care composition of claim 2, wherein said viscosity at said low shear rate is in the range of from about 50 Pas to about 1500 Pas; said shear rate index is in the range of from about 0.30 to about 0.50; and said viscosity at said high shear rate is in the range of from about 1 Pas to about 15 Pas.

13. The multi-phase personal care composition of claim 2, wherein a ratio of a first phase to a second phase is from about 90:10 to about 10:90.

14. The multi-phase personal care composition of claim 2, wherein at least one of said phases comprises at least one component selected from the group consisting of surfactants, thickeners, cationic surfactants, and fatty compounds.

15. The multi-phase personal care composition of claim 2, wherein at least one of said phases comprises at least one component selected from the group consisting of styling polymers, silicones, crosslinked silicone elastomers, peralkylene hydrocarbons, and hair coloring agents/dyes, anti-dandruff actives, humectants, water soluble nonionic polymers, cationic polymers, conditioning agents, and particles.

16. The multi-phase personal care composition of claim 2, wherein at least one of said phases is selected from the group consisting of a cleansing phase, a conditioning phase, a benefit phase, a high viscosity aqueous phase comprising a water thickener and water, and a high viscosity oil-in-water high internal phase emulsion comprising an oil and an aqueous carrier.

17. A method of delivering conditioning benefits to hair or skin, said method comprising the steps of:
a) topically applying an effective amount of a composition according to claim 2 onto said hair or skin; and
b) removing said composition from said hair or skin by rinsing with water.
